(19) United States
(12) Patent Application Publication
(10) Pub. No.: US 2019/0374945 A1
(43) Pub. Date: Dec. 12, 2019

(54) METHOD FOR ASSAYING BIOLOGICAL SAMPLE ON MICROFABRICATED CHIP

(71) Applicant: General Automation Lab Technologies Inc., San Carlos, CA (US)

(72) Inventor: Alexander Hallock, Redwood City, CA (US)

(21) Appl. No.: 16/434,131

(22) Filed: Jun. 6, 2019

Related U.S. Application Data
(60) Provisional application No. 62/681,910, filed on Jun. 7, 2018.

(51) Int. Cl. B01L 3/00; G01N 33/569

(52) U.S. Cl. CPC B01L 3/502715; B01L 3/502761; G01N 33/56911; B01L 2300/0825; B01L 2300/044; B01L 2300/0874; B01L 2300/0887; B01L 2300/168; B01L 2300/048; B01L 2300/0829

(57) ABSTRACT

A method of screening for at least one biological entity of interest in a sample using a microfabricated device. At least one cell from the sample and an amount of a nutrient is loaded into at least one microwell of the microfabricated device. A cover film containing a reagent is applied to the microfabricated device to retain the at least one cell. The microfabricated device is incubated to grow a plurality of cells from the at least one cell in the at least one microwell. During the incubation a gas may be produced that reacts with the reagent in the cover film, such that an optical property of an area of the cover film atop the at least one microwell can be affected. Based on the observed optical property, the presence of absence of at least one biological entity of interest can be determined.

### CROSS REFERENCE TO RELATED APPLICATIONS

This application claims the benefit of priority to U.S. provisional application No. 62/681,910, filed Jun. 7, 2018, the disclosure of which is incorporated herein by reference in its entirety. This application also relates to U.S. Nonprovisional patent application Ser. No. 15/135,377, filed on Apr. 21, 2016, the disclosure of which is incorporated by reference herein by its entirety.

### TECHNICAL FIELD

The present disclosure relates generally to innovations in microfabrication, microbiology, analytical chemistry. More specifically, the present disclosure relates to systems methods for high throughput screening and identification of biological entities on microfabricated devices.

### BACKGROUND

Identification and screening microorganisms in microbiomes has been gaining more attention and interest in the chemical, pharmaceutical, agricultural, and other industries as people realize the importance of microbiomes in our health, food production and environment. Useful information can be derived from such efforts, for example, for disease diagnosis and discovery of particular species of microorganisms having certain property of interest or can produce substances of interest.

Recently, various technology platforms utilizing plates or panels containing high density of wells for performing microorganism cultivation and screening have been developed. Due to the extremely small sizes of the wells, and small volume of material involved in each individual well, it is challenging to assay individual wells in-situ. Furthermore, to perform many assays, the contents of the wells need to be accessed and gathered, which disrupts the biological process ongoing in the wells. This often hinders, or even makes it impossible for the well contents to be used for other assays. Although prior to the assay, a replicate of the well contents can be made and transferred to another plate, such a step may require the use of sophisticated equipment and/or techniques, and could introduce errors.

### SUMMARY OF INVENTION

In some embodiments, a method of screening for at least one biological entity of interest in a sample using a microfabricated device having a top surface defining an array of microwells is provided. The method comprising: loading, into at least one microwell of the array of microwells, at least one cell from the sample and an amount of a nutrient; applying a cover film to the microfabricated device to retain the at least one cell in the at least one microwell, the cover film comprising a reagent; incubating the microfabricated device at predetermined conditions for a duration of time to grow a plurality of cells from the at least one cell in the at least one microwell; evaluating an optical property of an area of the cover film atop the at least one microwell, wherein if the plurality of cells grown in the incubation produces a gaseous compound that reacts with the reagent in the cover film to form an indicator compound, the optical property of the area of the cover film changes from that of the cover film in the absence of such reaction; and determining a presence or absence of at least one biological entity of interest in the at least one microwell based on the optical property.

In some embodiments of the method, the cover film can include a gas permeable membrane in direct contact with the top surface of the microfabricated device, and an outer layer laid on top of the gas permeable membrane. The reagent can be included in the outer layer. For example, the outer layer can include a polymeric substrate and the reagent dispersed or impregnated therein. The polymeric substrate can have a plurality of pores, into which the reagent can be loaded or attached.

In some embodiments, the reaction between the gaseous compound and the reagent produces a colorimetric change in the area the cover film.

In some embodiments, the nutrient includes a substrate that is a reactant of one or more chemical reactions that form the gaseous compound.

In some embodiments, the sample comprise a plurality of microbial cells of different species or genera, e.g., a microbiome sample. In some of these embodiments, the microbial cells include bacterial cells. In some embodiments, these bacterial cells are from a microbiome sample obtained from a specific environment, e.g., a soil, a human intestine, etc. Any microbial cell, e.g., bacterial cell, can be the biological entity of interest. In some embodiment, the at least one biological entity of interest comprises a eukaryotic cell.

In some embodiments, the gaseous compound can be one of hydrogen sulfide, oxygen, nitric oxide, carbon monoxide, or ammonia.

In some embodiments, the method further includes: if a biological entity of interest is determined to be present in the at least one microwell, transferring at least some of the plurality of cells after incubation to a target location.

In some embodiments, the at least one microwell includes a plurality of microwells, and loading the at least one cell comprises loading into each of the plurality of microwells, on average, a specific number of cell(s), for example, 1, 2, 3, 4, 5, 6, 7, 8, 9, 10, or more cells.

In some embodiments, each microwell of the array of microwells has a diameter of about 25 μm to about 500 μm.

In some embodiments, the surface density of the array of microwells is at least 750 microwells per cm².

In some embodiments, a distance between two neighboring microwells in the array of the microwells is less than 500 μm, or less than 250 μm, or less than 200 μm, or less than 150 μm, or less than 100 μm, or less than 80 μm, or less then 60 μm, or less than 50 μm, or less than 40 μm, or less than 30 μm, or less than 20 μm.

In some embodiments, evaluating the optical property can include: (a) measuring the optical property of the at least one microwell after the at least one cell and the nutrient have been loaded and before incubation; (b) measuring the optical property of the at least one microwell after incubation; and (c) comparing the measured optical property before incubation and after incubation.

In some embodiments, a method of screening for at least one biological entity of interest in a microbiome sample using a microfabricated device having a top surface defining an array of microwells is provided. Each microwell of the array of microwells has a diameter of about 25 μm to about 500 μm, and the surface density of the array of microwells is at least 750 microwells per cm². The method comprises: loading, into at least one microwell of the array of microwells, a portion of the microbiome sample and an amount of a nutrient, such that the at least one microwell of the array of microwells contain at least one cell; applying a cover film to the microfabricated device to retain the at least one cell in the at least one microwell, the cover film comprising a gas permeable membrane in direct contact with the top surface of the microfabricated device and an outer layer comprising a polymeric substrate and a reagent; incubating the microfabricated device at predetermined conditions for a duration of time to grow a plurality of cells in the at least one microwell; evaluating an optical property of areas of the cover film atop each of the array of microwells, wherein if the cells in the at least one microwell produce a gaseous compound that reacts with the reagent in the cover film to form an indicator compound, the optical property of the area of the cover film atop the corresponding microwell changes from that of the cover film in the absence of such reaction; and determining a presence or absence of at least one biological entity of interest in the at least one microwell based on the optical property. The sample may comprise bacterial cells, eukaryotic cells, etc.

In some embodiments, a method of screening for at least one biological entity of interest in a sample using a microfabricated device having a top surface defining an array of microwells, is provided. The method comprises: loading, into at least one microwell of the array of microwells, at least one cell from the sample; applying a cover film to the microfabricated device to retain the at least one cell in the at least one microwell, the cover film comprising a gas permeable membrane in direct contact with the top surface of the microfabricated device, and an outer layer laid on top of the gas permeable membrane, the outer layer comprising a polymeric substrate and a reagent; evaluating an optical property of an area of the cover film atop the at least one microwell, wherein if the at least one cell produces a gaseous compound that reacts with the reagent in the cover film to form an indicator compound, the optical property of the area of the cover film changes from that of the cover film in the absence of such reaction; and determining a presence or absence of at least one biological entity of interest in the at least one microwell based on the optical property. The method can further include growing a plurality of cells from the at least one cell before detection (e.g., before evaluating the optical property). In this manner, the time for obtaining an observable result may be shortened because of the proliferation and enrichment of the cells producing the gaseous compound.

In some embodiments, a method of screening for at least one biological entity of interest in a sample using a microfabricated device having a top surface defining an array of microwells is provided. The method comprising: loading, into at least one microwell of the array of microwells, at least one cell from the sample; applying a cover film to the microfabricated device to retain the at least one cell in the at least one microwell, the cover film comprising a gas permeable membrane in direct contact with the top surface of the microfabricated device; detecting a gaseous compound that has escaped from the at least one microwell through the membrane; and based on the detection, determining a presence or absence of at least one biological entity of interest in the at least one microwell based on the optical property. The cover film can further include an impregnated reagent, and the detecting can be done by evaluating an optical property of an area of the cover film atop the at least one microwell, wherein if a gaseous compound escapes the at least one microwell and reacts with the reagent in the cover film to form an indicator compound, the optical property of the area of the cover film changes from that of the cover film in the absence of such reaction. The reagent can be impregnated in a polymeric outer layer laid on the gas permeable membrane.

### BRIEF DESCRIPTION OF THE DRAWINGS

FIG. 1 is a perspective view illustrating a microfabricated device or chip in accordance with some embodiments.

FIGS. 2A-2C are top, side, and end views, respectively, illustrating dimensions of microfabricated device or chip in accordance with some embodiments.

FIGS. 3A and 3B are exploded and top views, respectively, illustrating a microfabricated device or chip in accordance with some embodiments.

FIG. 4 is a diagram illustrating a setup for performing an assay on the contents of microwells based on a gas produced in accordance with some embodiments.

### DETAILED DESCRIPTION

The present disclosure relates generally to systems and methods for isolation, culturing, sampling, and/or screening of biological entities. A microfabricated device (or a “chip”) is used for receiving a sample comprising at least one biological entity (e.g., at least one cell). The term “biological entity” may include, but is not limited to, an organism, a cell, a cell component, a cell product, and a virus, and the term “species” may be used to describe a unit of classification, including, but not limited to, an operational taxonomic unit (OTU), a genotype, a phylotype, a phenotype, an ecotype, a history, a behavior or interaction, a product, a variant, and an evolutionarily significant unit.

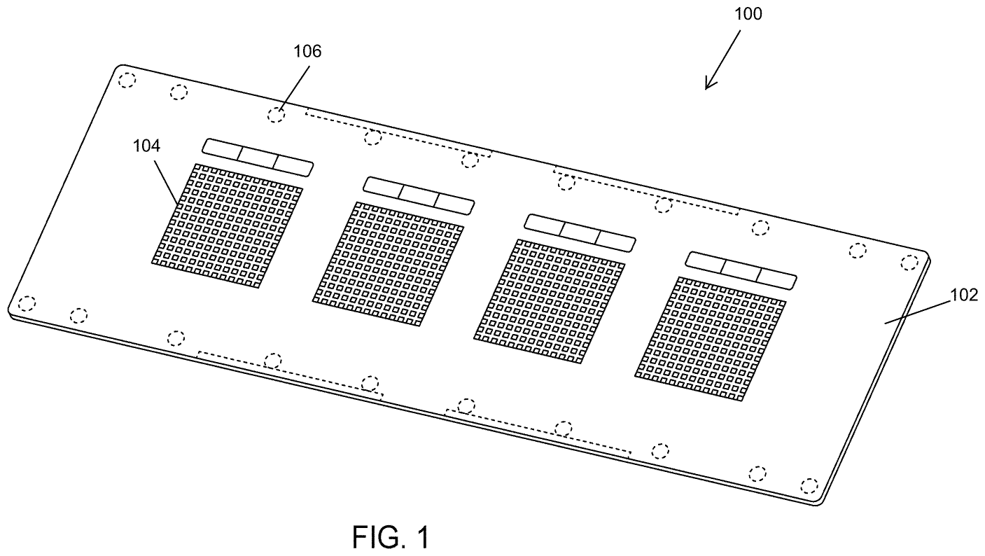

As used herein, a microfabricated device or chip may define a high density array of microwells (or experimental units). For example, a microfabricated chip comprising a “high density” of microwells may include about 150 microwells per cm² to about 160,000 microwells or more per cm² (for example, at least 150 microwells per cm², at least 250 microwells per cm², at least 400 microwells per cm², at least 500 microwells per cm², at least 750 microwells per cm², at least 1,000 microwells per cm², at least 2,500 microwells per cm², at least 5,000 microwells per cm², at least 7,500 microwells per cm², at least 10,000 microwells per cm², at least 50,000 microwells per cm², at least 100,000 microwells per cm², or at least 160,000 microwells per cm²). A substrate of a microfabricated chip may include about or more than 10,000,000 microwells or locations. For example, an array of microwells may include at least 96 locations, at least 1,000 locations, at least 5,000 locations, at least 10,000 locations, at least 50,000 locations, at least 100,000 locations, at least 500,000 locations, at least 1,000,000 locations, at least 5,000,000 locations, or at least 10,000,000 locations. The arrays of microwells may form grid patterns, and be grouped into separate areas or sections. The dimensions of a microwell may range from nanoscopic (e.g., a diameter from about 1 to about 100 nanometers) to microscopic. For example, each microwell may have a diameter of about 1 μm to about 800 μm, a diameter of about 25 μm to about 500 μm, or a diameter of about 30 μm to about 100 μm. A microwell may have a diameter of about or less than 1 μm, about or less than 5 μm, about or less than 10 μm, about or less than 25 μm, about or less than 50 μm, about or less than 100 μm, about or less than 200 μm, about or less than 300 μm, about or less than 400 μm, about or less than 500 μm, about or less than 600 μm, about or less than 700 μm, or about or less than 800 μm. In exemplary embodiments, the diameter of the microwells can be about 100 μm or smaller, or 50 μm or smaller. A microwell may have a depth of about 25 μm to about 100 μm, e.g., about 1 μm, about 5 μm, about 10 μm, about 25 μm, about 50 μm, about 100 μm. It can also have greater depth, e.g., about 200 μm, about 300 μm, about 400 μm, about 500 μm. The microfabricated chip can have two major surfaces: a top surface and a bottom surface, where the microwells have openings at the top surface. Each microwell of the microwells may have an opening or cross section having any shape, e.g., round, hexagonal, square, or other shapes. Each microwell may include sidewalls. For microwells that are not round in their openings or cross sections, the diameter of the microwells described herein refer to the effective diameter of a circular shape having an equivalent area. For example, for a square shaped microwell having side lengths of 10×10 microns, a circle having an equivalent area (100 square microns) has a diameter of 11.3 microns. Each microwell may include a sidewall or sidewalls. The sidewalls may have a cross-sectional profile that is straight, oblique, and/or curved. Each microwell includes a bottom which can be flat, round, or of other shapes. The microfabricated chip (with the microwells thereon) may be manufactured from a polymer, e.g., a cyclic olefin polymer, via precision injection molding or some other process such as embossing. The chip may have a substantially planar major surface. FIG. 1 shows a schematic depiction of a microfabricated chip, whose edges are generally parallel to the directions of the rows and the columns of the microwells on the chip.

The high density microwells on the microfabricated chip can be used to conduct various experiments, such as growth or cultivation or screening of various species of bacteria and other microorganisms (or microbes) such as aerobic, anaerobic, and/or facultative aerobic microorganisms. The microwells may be used to conduct experiments with eukaryotic cells such as mammalian cells. Also, the microwells can be used to conduct various genomic or proteomic experiments, and may contain cell products or components, or other biological substances or entities, such as a cell surface (e.g., a cell membrane or wall), a metabolite, a vitamin, a hormone, a neurotransmitter, an antibody, an amino acid, an enzyme, a protein, a saccharide, ATP, a lipid, a nucleoside, a nucleotide, a nucleic acid (e.g., DNA or RNA), etc.

A cell may be Archaea, Bacteria, or Eukaryota (e.g., fungi). For example, a cell may be a microorganism, such as an aerobic, anaerobic, or facultative aerobic microorganisms. A virus may be a bacteriophage. Other cell components/products may include, but are not limited to, proteins, amino acids, enzymes, saccharides, adenosine triphosphate (ATP), lipids, nucleic acids (e.g., DNA and RNA), nucleosides, nucleotides, cell membranes/walls, flagella, fimbriae, organelles, metabolites, vitamins, hormones, neurotransmitters, and antibodies.

A nutrient may be defined (e.g., a chemically defined or synthetic medium) or undefined (e.g., a basal or complex medium). A nutrient may include or be a component of a laboratory-formulated and/or a commercially manufactured medium (e.g., a mix of two or more chemicals). A nutrient may include or be a component of a liquid nutrient medium (i.e., a nutrient broth), such as a marine broth, a lysogeny broth (e.g., Luria broth), etc. A nutrient may include or be a component of a liquid medium mixed with agar to form a solid medium and/or a commercially available manufactured agar plate, such as blood agar.

A nutrient may include or be a component of selective media. For example, selective media may be used for the growth of only certain biological entities or only biological entities with certain properties (e.g., antibiotic resistance or synthesis of a certain metabolite). A nutrient may include or be a component of differential media to distinguish one type of biological entity from another type of biological entity or other types of biological entities by using biochemical characteristics in the presence of specific indicator (e.g., neutral red, phenol red, eosin y, or methylene blue).

A nutrient may include or be a component of an extract of or media derived from a natural environment. For example, a nutrient may be derived from an environment natural to a particular type of biological entity, a different environment, or a plurality of environments. The environment may include, but is not limited to, one or more of a biological tissue (e.g., connective, muscle, nervous, epithelial, plant epidermis, vascular, ground, etc.), a biological fluid or other biological product (e.g., amniotic fluid, bile, blood, cerebrospinal fluid, cerumen, exudate, fecal matter, gastric fluid, interstitial fluid, intracellular fluid, lymphatic fluid, milk, mucus, rumen content, saliva, sebum, semen, sweat, urine, vaginal secretion, vomit, etc.), a microbial suspension, air (including, e.g., different gas contents), supercritical carbon dioxide, soil (including, e.g., minerals, organic matter, gases, liquids, organisms, etc.), sediment (e.g., agricultural, marine, etc.), living organic matter (e.g., plants, insects, other small organisms and microorganisms), dead organic matter, forage (e.g., grasses, legumes, silage, crop residue, etc.), a mineral, oil or oil products (e.g., animal, vegetable, petrochemical), water (e.g., naturally-sourced freshwater, drinking water, seawater, etc.), and/or sewage (e.g., sanitary, commercial, industrial, and/or agricultural wastewater and surface runoff).

FIG. 1 is a perspective view illustrating a microfabricated device or chip in accordance with some embodiments. Chip 100 includes a substrate shaped in a microscope slide format with injection-molded features on top surface 102. The features include four separate microwell arrays (or microarrays) 104 as well as ejector marks 106. The microwells in each microarray are arranged in a grid pattern with well-free margins around the edges of chip 100 and between microarrays 104.

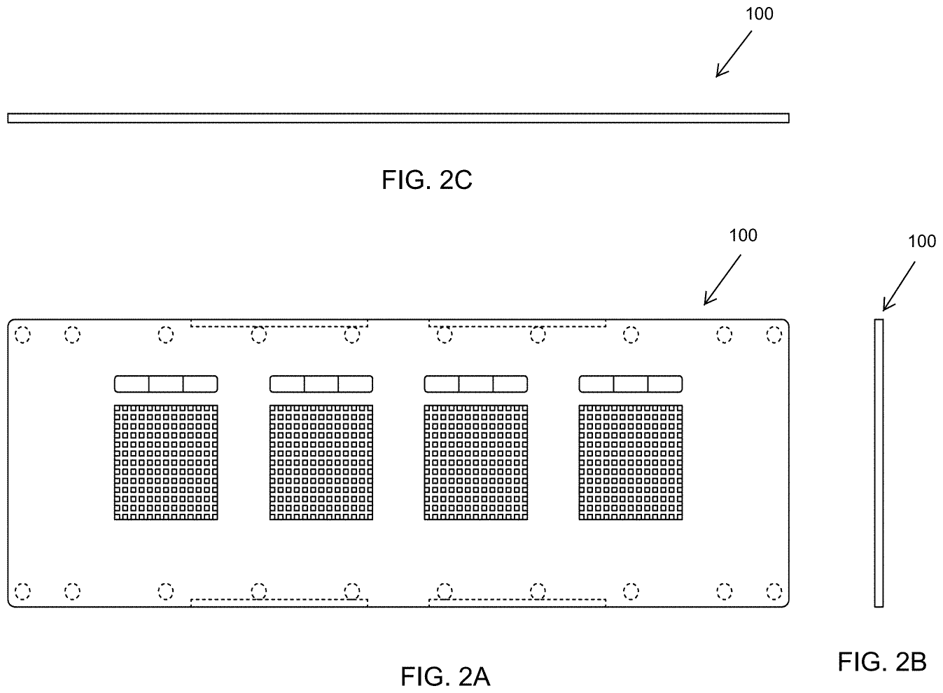

FIGS. 2A-2C are top, side, and end views, respectively, illustrating dimensions of chip 100 in accordance with some embodiments. In FIG. 2A, the top of chip 100 is approximately 25.5 mm by 75.5 mm. In FIG. 2B, the end of chip 100 is approximately 25.5 mm by 0.8 mm. In FIG. 2C, the side of chip 100 is approximately 75.5 mm by 0.8 mm.

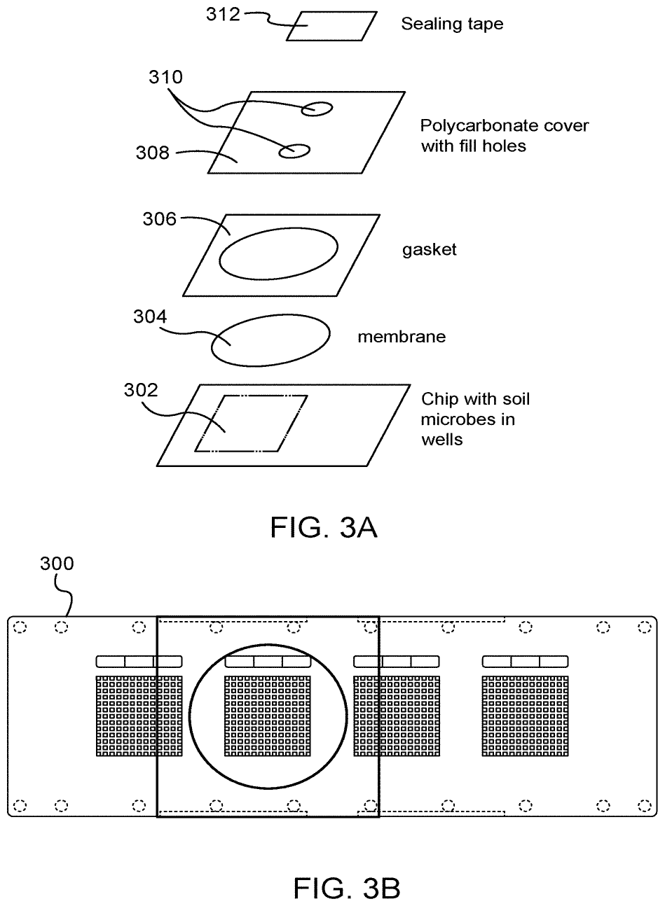

After a sample is loaded on a microfabricated device, a membrane may be applied to at least a portion of a microfabricated device. FIG. 3A is an exploded diagram of the microfabricated device 300 shown from a top view in FIG. 3B in accordance with some embodiments. Device 300 includes a chip with an array of wells 302 holding, for example, soil microbes. A membrane 304 is placed on top of the array of wells 302. A gasket 306 is placed on top of the membrane 304. A polycarbonate cover 308 with fill holes 310 is placed on top of the gasket 306. Finally, sealing tape 312 is applied to the cover 308.

A membrane may cover at least a portion of a microfabricated device including one or more experimental units, wells, or microwells. For example, after a sample is loaded on a microfabricated device, at least one membrane may be applied to at least one microwell of a high density array of microwells. A plurality of membranes may be applied to a plurality of portions of a microfabricated device. For example, separate membranes may be applied to separate subsections of a high density array of microwells.

A membrane may be connected, attached, partially attached, affixed, sealed, and/or partially sealed to a microfabricated device to retain at least one biological entity in the at least one microwell of the high density array of microwells. For example, a membrane may be reversibly affixed to a microfabricated device using lamination. A membrane may be punctured, peeled back, detached, partially detached, removed, and/or partially removed to access at least one biological entity in the at least one microwell of the high density array of microwells.

A portion of the population of cells in at least one experimental unit, well, or microwell may attach to a membrane (via, e.g., adsorption). If so, the population of cells in at least one experimental unit, well, or microwell may be sampled by peeling back the membrane such that the portion of the population of cells in the at least one experimental unit, well, or microwell remains attached to the membrane.

A membrane may be impermeable, semi-permeable, selectively permeable, differentially permeable, and/or partially permeable to allow diffusion of at least one nutrient into the at least one microwell of a high density array of microwells. For example, a membrane may include a natural material and/or a synthetic material. A membrane may include a hydrogel layer and/or filter paper. In some embodiments, a membrane is selected with a pore size small enough to retain at least some or all of the cells in a microwell. For mammalian cells, the pore size may be a few microns and still retain the cells. However, in some embodiments, the pore size may be less than or equal to about 0.2 μm, such as 0.1 μm. An impermeable membrane has a pore size approaching zero. It is understood that the membrane may have a complex structure that may or may not have defined pore sizes

In one aspect of the present invention, there is provided a nondestructive, spatially-sensitive assay of a biological entity of interest of the samples loaded in the microfabricated chip based on a gaseous substance produced in individual wells. The assay can be performed in a highly parallelized manner, enabling fast screening of very large numbers of microbes (e.g. microbiomes) in a single experiment. Compared with alternative methods for assaying contents of microwells, which may involve adding reagents into the well contents, and therefore cause disruption of the normal cell growth/proliferation or even destroy the cells in the microwells (a lot of reagents are harmful or toxic to the cells), the method of the present disclosure separates the site of detection (in the cover film atop of the microwell) from the well contents, therefore does not interfere with cell growth or proliferation in the microwells. This allows the well contents to be available for further assays or tests.

In some embodiments, a method of screening for at least one biological entity of interest in a sample is provided. At least one microwell (and preferably a plurality of microwells) of the array of microwells on a microfabricated chip is loaded with at least one cell from the sample and an amount of a nutrient. A cover film containing a reagent is applied to the microfabricated device to retain the at least one cell in the at least one microwell. The microfabricated device can be incubated at predetermined conditions for a duration of time to grow a plurality of cells from the at least one cell in the at least one microwell. An optical property of an area of the cover film atop the at least one microwell is evaluated. If the at least one cell or the plurality of cells grown in the incubation produces a gaseous compound or substance that reacts with the reagent in the cover film to form an indicator compound, the optical property of the area of the cover film changes from that of the cover film in the absence of such reaction. Based on the evaluated optical property, a presence or absence of at least one biological entity of interest in the at least one microwell is determined.

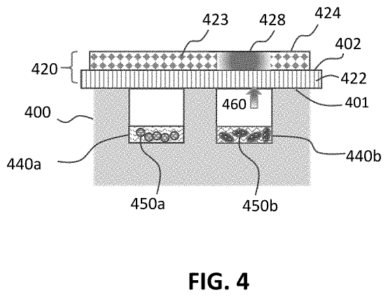

FIG. 4 is a schematic depiction of an example setup for performing an assay of the contents of microwells based on gases produced from the microwells. As shown in FIG. 4, the cover film 420 can include a gas permeable membrane 422 that allows oxygen, carbon dioxide, or other gases of interest to come in and/or out of a microwell but retains solid and liquid contents of the microwell. The gas permeable membrane can have a top (or major) surface 402, and can be applied on and in direct contact with the top surface 401 of the microfabricated device 400 as a seal to retain the contents of the microwells 440a and 440b. It is understood that different gases would have different molecular sizes and affinity to different membrane materials, and their transport rates or behavior through the membrane may also depend on other microstructural parameters of the membrane (which in turn may depend on how the membrane is manufactured, e.g., the cast and drying process, how the pores are created inside the membrane, etc.). The overall permeability of the membrane with respect to any particular gas also depends on the thickness of the membrane. The particular membrane (e.g., with specific permeability, thickness and other parameters) selected for any assay can be determined based on the nature of the assay, as well as the identity and amount of the gas compounds expected to be produced by the microwell contents. In some examples, the gas permeable membrane can be polyurethane sealing membrane with FDA-approved acrylic adhesive commonly used to seal multi-well plates.

In some embodiments, the gas permeable membrane can have a microstructure that enables an anisotropic diffusion of the gaseous compound. The microstructure of the membrane can be such that the gaseous substances can diffuse more rapidly along the thickness direction of the membrane (in other words, perpendicular to the top surface 401 of the microfabricated chip 400) than along its transverse direction (or the direction parallel to the top surface 401). As an example, the gas permeable membrane can have a plurality through channels that are substantially normal to the major surface of membrane. In such a structure, gas diffusion will be substantially along the direction normal to the membrane surface, and lateral diffusion along the membrane is negligible. As a result, cross contamination between closely spaced wells can be reduced or avoided.

The reagent located above the permeable membrane can be a chemical compound or a composition comprising more than one chemical compounds, which when reacting with a gas compound of interest, produce an indicator compound that possess an optical property amenable for detection by direct visual observation by naked eye or by optical instruments (e.g., UV, visible light, or fluorescence instrument) at appropriate wavelength. The intensity of such optical property can be proportional to the amount of the indicator compound produced, which in turn relates to the amount of gas compound produced by the well contents during incubation/growth.

As illustrated in FIG. 4, the reagent 423 can be dispersed in a reagent layer 424 applied on top of the gas permeable membrane 422. As an illustration, the well contents 450a in well 440a does not give off any gas, whereas the well contents 450b in well 440b gives off a gas 460, which permeates through the gas permeable membrane 422 and causes an observable change in optical property, e.g., a colorimetric change in an area 428 of the reagent layer which is on top of the well 440b. The observable change could also be a change in optical property of the cover film such as fluorescence or chemiluminescence. The extent of the change in the optical property may be proportionate to, or in other positive correlation with the amount of the gas released. The gas compound to be detected needs to be volatile enough to escape from the microwell, permeate through the gas permeable membrane portion above the microwell, and into the detection region on the reagent layer. The reagent can be selected for different gas compounds of interest, for example:

| gas compound to be | | color change |
|---|---|---|
| detected | reagent | expected |
| H₂S | Ag or Ag⁺ | dark brown |
| NO | Griess agent | pink/red |
| NH₃ | Berthelot's reagent | blue |
| CO | Palladium chloride | red |
| O₂ | Indigo-carmine | blue |
| HCN | Tris(1,10-phenanthroline)-Fe(II) | red |

As shown in FIG. 4, the layer 424 is continuous and covers two adjacent wells 440. But it is understood that the layer 424 can be a continuous layer covering the entire top surface of the gas permeable membrane (which can seal hundreds or thousands of wells of the chip at once) or any selected area(s) of the gas permeable membrane. For example, the reagent layer can include many discrete portions or patches each separately covering an area of the gas membrane on top of each individual well.

The reagent can be embedded or entrained in the reagent layer during the preparation process of the reagent layer. For example, it can be in the form of micro or nano particles trapped or impregnated in micropores of the reagent layer. In one example, a reagent impregnating layer was prepared as follows: a sulfonated tetrafluoroethylene based fluoropolymer-copolymer Nafion® powder was mixed with alcohol, AgNO₃ aqueous solution (added dropwise), water, and glycerol in a certain ratio to make a viscous liquid and applied in any desired location or pattern on top of a gas permeable membrane, e.g., a breathe-easy membrane having a thickness of about 25 microns, and allowed to dry/cure. Curing can be done at room temperature or in an oven up to 80 degrees C. and for some time between an hour and overnight. The cured reagent layer includes a plurality of pores with Ag (or Ag+) loaded therein. The gas permeable membrane with the cured reagent layer constitutes the cover film and was applied on top of a microfabricated device to seal a plurality of wells, some of which were preloaded with sulfate reducing bacteria (SRB) Desulfovibrio and an amount of nutrient containing sulfate while some other wells were preloaded with control (other bacteria than SRB). Overnight, the areas on the membrane corresponding to those wells loaded with SRB showed clear change of color, while the areas on the membrane corresponding to the control wells did not show change of color. The cover film was removed and cut into small squares and imaged under a microscope, and it was observed that color change was localized to an area just a little larger than a well and changes through the entire thickness of the polymer. In this case, the SRB reduced the sulfate in the nutrient provided in the microwells, which produced H₂S as a product, which permeates through the membrane and reacted with the silver ions in the reagent layer to form Ag₂S.

Other ionic polymer can be used as polymeric substrate for the reagent layer. The change of color (or other change of optical property) of the reagent layer can be observed continuously or periodically to obtain a time-dependent curve of the optical property so as to better monitor the progress of the reactions within the microwells. The microfabricated chip can be loaded with a sample containing a plurality of species, strains or genera of microorganisms or eukaryotic cells, and the cells can be loaded such that each of the plurality of microwells can contain, on average, one cell, two cells, three cells, four cells, five cells, six cells, seven cells, eight cells, and so on. This can be accomplished using a cell sorter or other available techniques.

While in some of the embodiments described herein, a reagent contained in the cover film is used for reaction with the gas released from the microwells, alternative methods can be used to directly test the gas without using a reagent contained in the cover film. For example, the escaped gas may be detected by mass spectroscopy with a high resolution ionization system which allows for detection of ion species formed from the escaped gas from individual microwells with a resolution sufficient to differentiate from microwell to microwell.

While various embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed.

What is claimed is:

1. A method of screening for at least one biological entity of interest in a sample using a microfabricated device having a top surface defining an array of microwells, the method comprising:
loading, into at least one microwell of the array of microwells, at least one cell from the sample and an amount of a nutrient;
applying a cover film to the microfabricated device to retain the at least one cell in the at least one microwell, the cover film comprising a reagent;
incubating the microfabricated device at predetermined conditions for a duration of time to grow a plurality of cells from the at least one cell in the at least one microwell;
evaluating an optical property of an area of the cover film atop the at least one microwell, wherein if the plurality of cells grown in the incubation produces a gaseous compound that reacts with the reagent in the cover film to form an indicator compound, the optical property of the area of the cover film changes from that of the cover film in the absence of such reaction; and
determining a presence or absence of at least one biological entity of interest in the at least one microwell based on the optical property.

2. The method of claim 1, wherein the cover film comprises a gas permeable membrane in direct contact with the top surface of the microfabricated device, and an outer layer laid on top of the gas permeable membrane, the outer layer comprising a polymeric substrate and the reagent.

3. The method of claim 1, wherein the reaction between the gaseous compound and the reagent produces a colorimetric change in the area the cover film.

4. The method of claim 1, wherein the nutrient includes a substrate that is a reactant of one or more chemical reactions that form the gaseous compound.

5. The method of claim 1, wherein the sample comprise a plurality of microbial cells of different species or genera.

6. The method of claim 2, wherein the polymeric substrate is a porous ionic polymer having a plurality of pores.

7. The method of claim 6, wherein the reagent is loaded in the pores of the polymeric substrate.

8. The method of claim 1, wherein the gaseous compound is selected from hydrogen sulfide, oxygen, carbon monoxide, nitric oxide, and ammonia.

9. The method of claim 1, wherein the at least one biological entity of interest comprises a eukaryotic cell.

10. The method of claim 1, wherein the at least one biological entity of interest comprises bacteria.

11. The method of claim 1, further comprising:
if a biological entity of interest is determined to be present in the at least one microwell, transferring at least some of the plurality of cells after incubation to a target location.

12. The method of claim 1, wherein the at least one microwell includes a plurality of microwells, and wherein loading the at least one cell comprises loading into each of the plurality of microwells, on average, one cell.

13. The method of claim 1, wherein each microwell of the array of microwells has a diameter of about 25 μm to about 500 μm.

14. The method of claim 1, wherein the surface density of the array of microwells is at least 750 microwells per cm².

15. The method of claim 1, wherein a distance between two neighboring microwells in the array of the microwells is less than 500 μm.

16. The method of claim 1, wherein evaluating the optical property comprises:
(a) measuring the optical property of the at least one microwell after the at least one cell and the nutrient have been loaded and before incubation;
(b) measuring the optical property of the at least one microwell after incubation; and
(c) comparing the measured optical property before incubation and after incubation.

17. A method of screening for at least one biological entity of interest in a microbiome sample using a microfabricated device having a top surface defining an array of microwells, wherein each microwell of the array of microwells has a diameter of about 25 μm to about 500 μm, and wherein the surface density of the array of microwells is at least 750 microwells per cm², the method comprising:
loading, into at least one microwell of the array of microwells, a portion of the microbiome sample and an amount of a nutrient, such that the at least one microwell of the array of microwells contain at least one cell;
applying a cover film to the microfabricated device to retain the at least one cell in the at least one microwell, the cover film comprising a gas permeable membrane in direct contact with the top surface of the microfabricated device and an outer layer comprising a polymeric substrate and a reagent;
incubating the microfabricated device at predetermined conditions for a duration of time to grow a plurality of cells in the at least one microwell;
evaluating an optical property of areas of the cover film atop each of the array of microwells, wherein if the cells in the at least one microwell produce a gaseous compound that reacts with the reagent in the cover film to form an indicator compound, the optical property of the area of the cover film atop the corresponding microwell changes from that of the cover film in the absence of such reaction; and
determining a presence or absence of at least one biological entity of interest in the at least one microwell based on the optical property.

18. The method of claim 17, wherein the at least cell comprises a bacterial cell.

19. A method of screening for at least one biological entity of interest in a sample using a microfabricated device having a top surface defining an array of microwells, the method comprising:
loading, into at least one microwell of the array of microwells, at least one cell from the sample;
applying a cover film to the microfabricated device to retain the at least one cell in the at least one microwell, the cover film comprising a gas permeable membrane in direct contact with the top surface of the microfabricated device, and an outer layer laid on top of the gas permeable membrane, the outer layer comprising a polymeric substrate and a reagent;
evaluating an optical property of an area of the cover film atop the at least one microwell, wherein if the at least one cell produces a gaseous compound that reacts with the reagent in the cover film to form an indicator compound, the optical property of the area of the cover film changes from that of the cover film in the absence of such reaction; and
determining a presence or absence of at least one biological entity of interest in the at least one microwell based on the optical property.

20. The method of claim 19, further comprising:
growing a plurality of cells from the at least one cell.

21. A method of screening for at least one biological entity of interest in a sample using a microfabricated device having a top surface defining an array of microwells, the method comprising:
loading, into at least one microwell of the array of microwells, at least one cell from the sample;
applying a cover film to the microfabricated device to retain the at least one cell in the at least one microwell, the cover film comprising a gas permeable membrane in direct contact with the top surface of the microfabricated device;
detecting a gaseous compound that has escaped from the at least one microwell through the membrane; and
based on the detection, determining a presence or absence of at least one biological entity of interest in the at least one microwell based on the optical property.

22. The method of claim 21, wherein the cover film further comprises an impregnated reagent, and wherein the detecting comprises evaluating an optical property of an area of the cover film atop the at least one microwell, wherein if a gaseous compound escapes the at least one microwell and reacts with the reagent in the cover film to form an indicator compound, the optical property of the area of the cover film changes from that of the cover film in the absence of such reaction.

23. The method of claim 22, wherein the reagent is impregnated in a polymeric outer layer laid on the gas permeable membrane.
